(19) United States
(12) Patent Application Publication
(10) Pub. No.: US 2004/0033599 A1
(43) Pub. Date: Feb. 19, 2004

(54) Medium for preparing dedifferentiated cells

(76) Inventor: Lawrence Rosenberg, Cote St-Luc (CA)

Correspondence Address: NIXON PEABODY LLP, ATTENTION: DAVID RESNICK, 101 FEDERAL STREET, BOSTON, MA, 02110, US

(21) Appl. No.: 10/426,255

(22) Filed: Apr. 29, 2003

Related U.S. Application Data
(63) Continuation-in-part of application No. 10/111,485, filed on Aug. 7, 2002.
Application No. 10/111,485 is a 371 of international application No. PCT/CA00/01284, filed on Oct. 27, 2000.
(60) Provisional application No. 60/162,137, filed on Oct. 29, 1999.

(51) Int. Cl. C12N 5/00; C12N 5/02; C12N 5/08

(52) U.S. Cl. 435/377; 435/366; 435/373; 435/383

(57) ABSTRACT

The present invention relates to a medium for preparing dedifferentiated cells derived from post-natal islets of Langerhans. The medium comprises in a physiologically acceptable culture medium an effective amount of a solid matrix environment for a three-dimensional culture, a soluble matrix protein, and a first and a second factor for developing, maintaining and expanding the dedifferentiated cells. Such a medium may be used in an in vitro method for islet cell expansion.

### RELATED APPLICATIONS

This application is a continuation-in-part of application Ser. No. 10/111,485 filed on Apr. 25, 2002 which is still pending and which is a National Phase entry of International application number PCT/CA00/01284 filed on Oct. 27, 2000, now abandoned, and which is claiming the benefit of priority of application serial No. 60/162,137 filed on Oct. 29, 1999 and which is now abandoned and all above applications are all incorporated by reference.

### BACKGROUND OF THE INVENTION

(a) Field of the Invention

The invention relates to a medium for preparing dedifferentiated cells and more particularly to a basal feeding medium for the development, maintenance and expansion of a dedifferentiated cell population with at least bipotentiality, which may be used in an in vitro method for islet cell expansion.

(b) Description of Prior Art

Diabetes Mellitus

Diabetes mellitus has been classified as type I, or insulin-dependent diabetes mellitus (IDDM) and type II, or non-insulin-dependent diabetes mellitus (NIDDM). NIDDM patients have been subdivided further into (a) nonobese (possibly IDDM in evolution), (b) obese, and (c) maturity onset (in young patients). Among the population with diabetes mellitus, about 20% suffer from IDDM. Diabetes develops either when a diminished insulin output occurs or when a diminished sensitivity to insulin cannot be compensated for by an augmented capacity for insulin secretion. In patients with IDDM, a decrease in insulin secretion is the principal factor in the pathogenesis, whereas in patients with NIDDM, a decrease in insulin sensitivity is the primary factor. The mainstay of diabetes treatment, especially for type I disease, has been the administration of exogenous insulin.

Rationale for More Physiologic Therapies

Tight glucose control appears to be the key to the prevention of the secondary complications of diabetes. The results of the Diabetes Complications and Control Trial (DCCT), a multicenter randomized trial of 1441 patients with insulin dependent diabetes, indicated that the onset and progression of diabetic retinopathy, nephropathy, and neuropathy could be slowed by intensive insulin therapy (The Diabetes Control and Complication Trial Research Group, N. Engl. J. Med., 1993; 29:977-986). Strict glucose control, however, was associated with a three-fold increase in incidence of severe hypoglycemia, including episodes of seizure and coma. As well, although glycosylated hemoglobin levels decreased in the treatment group, only 5% maintained an average level below 6.05% despite the enormous amount of effort and resources allocated to the support of patients on the intensive regime (The Diabetes Control and Complication Trial Research Group, N. Engl. J. Med., 1993; 29:977-986). The results of the DCCT clearly indicated that intensive control of glucose can significantly reduce (but not completely protect against) the long-term microvascular complications of diabetes mellitus.

Other Therapeutic Options

The delivery of insulin in a physiologic manner has been an elusive goal since insulin was first purified by Banting, Best, McLeod and Collip. Even in a patient with tight glucose control, however, exogenous insulin has not been able to achieve the glucose metabolism of an endogenous insulin source that responds to moment-to-moment changes in glucose concentration and therefore protects against the development of microvascular complications over the long term.

A major goal of diabetes research, therefore, has been the development of new forms of treatment that endeavor to reproduce more closely the normal physiologic state. One such approach, a closed-loop insulin pump coupled to a glucose sensor, mimicking β-cell function in which the secretion of insulin is closely regulated, has not yet been successful. Only total endocrine replacement therapy in the form of a transplant has proven effective in the treatment of diabetes mellitus. Although transplants of insulin-producing tissue are a logical advance over subcutaneous insulin injections, it is still far from clear whether the risks of the intervention and of the associated long-term immunosuppressive treatment are lower those in diabetic patients under conventional treatment.

Despite the early evidence of the potential benefits of vascularized pancreas transplantation, it remains a complex surgical intervention, requiring the long-term administration of chronic immunosuppression with its attendant side effects. Moreover, almost 50% of successfully transplanted patients exhibit impaired tolerance curves (Wright F H et al., Arch. Surg., 1989;124:796-799; Landgraft R et al., Diabetologia 1991; 34 (suppl 1):S61; Morel P et al., Transplantation 1991; 51:990-1000), raising questions about their protection against the long-term complications of chronic hyperglycemia.

The major complications of whole pancreas transplantation, as well as the requirement for long term immunosuppression, has limited its wider application and provided impetus for the development of islet transplantation. Theoretically, the transplantation of islets alone, while enabling tight glycemic control, has several potential advantages over whole pancreas transplantation. These include the following: (i) minimal surgical morbidity, with the infusion of islets directly into the liver via the portal vein; (ii) the possibility of simple re-transplantation for graft failures; (iii) the exclusion of complications associated with the exocrine pancreas; (iv) the possibility that islets are less immunogenic, eliminating the need for immunosuppression and enabling early transplantation into non-uremic diabetics; (v) the possibility of modifying islets in vitro prior to transplantation to reduce their immunogenicity; (vi) the ability to encapsulate islets in artificial membranes to isolate them from the host immune system; and (vii) the related possibility of using xenotransplantation of islets immunoisolated as part of a biohybrid system. Moreover, they permit the banking of the endocrine cryopreserved tissue and a careful and standardized quality control program before the implantation.

The Problem of Islet Transplantation

Adequate numbers of isogenetic islets transplanted into a reliable implantation site can only reverse the metabolic abnormalities in diabetic recipients in the short term. In those that were normoglycemic post-transplant, hyperglycemia recurred within 3-12 mo. (Orloff M, et al., Transplantation 1988; 45:307). The return of the diabetic state that occurs with time has been attributed either to the ectopic location of the islets, to a disruption of the enteroinsular axis, or to the transplantation of an inadequate islet cell mass (Bretzel R G, et al. In: Bretzel R G, (ed) Diabetes mellitus (Berlin: Springer, 1990) p.229).

Studies of the long term natural history of the islet transplant, that examine parameters other than graft function, are few in number. Only one report was found in which an attempt was specifically made to study graft morphology (Alejandro R, et al., J Clin Invest 1986; 78: 1339). In that study, purified islets were transplanted into the canine liver via the portal vein. During prolonged follow-up, delayed failures of graft function occurred. Unfortunately, the graft was only examined at the end of the study, and not over time as function declined. Delayed graft failures have also been confirmed by other investigators for dogs (Warnock G L et al., Can. J. Surg., 1988; 31: 421 and primates; Sutton R, et al., Transplant Proc., 1987; 19: 3525). Most failures are presumed to be the result of rejection despite appropriate immunosuppression.

Because of these failures, there is currently much enthusiasm for the immunoisolation of islets, which could eliminate the need for immunosuppression. The reasons are compelling. Immunosuppression is harmful to the recipient, and may impair islet function and possibly cell survival (Metrakos P, et al., J. Surg. Res., 1993; 54: 375). Unfortunately, micro-encapsulated islets injected into the peritoneal cavity of the dog fail within 6 months (Soon-Shiong P, et al., Transplantation 1992; 54: 769), and islets placed into a vascularized biohybrid pancreas also fail, but at about one year. In each instance, however, histological evaluation of the graft has indicated a substantial loss of islet mass in these devices (Lanza R P, et al., Diabetes 1992; 41: 1503). No reasons have been advanced for these changes. Therefore maintenance of an effective islet cell mass post-transplantation remains a significant problem.

In addition to this unresolved issue, is the ongoing problem of the lack of source tissue for transplantation. The number of human donors is insufficient to keep up with the potential number of recipients. Moreover, given the current state of the art of islet isolation, the number of islets that can be isolated from one pancreas is far from the number required to effectively reverse hyperglycemia in a human recipient.

In response, three competing technologies have been proposed and are under development. First, islet cryopreservation and islet banking. The techniques involved, though, are expensive and cumbersome, and do not easily lend themselves to widespread adoption. In addition, islet cell mass is also lost during the freeze-thaw cycle. Therefore this is a poor long-term solution to the problem of insufficient islet cell mass. Second, is the development of islet xenotransplantation. This idea has been coupled to islet encapsulation technology to produce a biohybrid implant that does not, at least in theory, require immunosuppression. There remain many problems to solve with this approach, not least of which, is that the problem of the maintenance of islet cell mass in the post-transplant still remains. Third, is the resort to human fetal tissue, which should have a great capacity to be expanded ex vivo and then transplanted. However, in addition to the problems of limited tissue availability, immunogenicity, there are complex ethical issues surrounding the use of such a tissue source that will not soon be resolved. However, there is an alternative that offers similar possibilities for near unlimited cell mass expansion.

An entirely novel approach, proposed by Rosenberg in 1995 (Rosenberg L et al., Cell Transplantation, 1995;4:371-384), was the development of technology to control and modulate islet cell neogenesis and new islet formation, both in vitro and in vivo. The concept assumed that (a) the induction of islet cell differentiation was in fact controllable; (b) implied the persistence of a stem cell-like cell in the adult pancreas; and (c) that the signal(s) that would drive the whole process could be identified and manipulated.

In as series of in vivo studies, Rosenberg and co-workers established that these concepts were valid in principle, in the in vivo setting (Rosenberg L et al., Diabetes, 1988;37:334-341; Rosenberg L et al., Diabetologia, 1996;39:256-262), and that diabetes could be reversed.

The well known teachings of in vitro islet cell expansion from a non-fetal tissue source comes from Peck and co-workers (Corneliu J G et al., Horm. Metab. Res., 1997;29:271-277), who describe isolation of a pluripotent stem cell from the adult mouse pancreas that can be directed toward an insulin-producing cell. These findings have not been widely accepted. First, the result has not proven to be reproducible. Second, the so-called pluripotential cells have never been adequately characterized with respect to phenotype. And third, the cells have certainly not been shown to be pluripotent.

More recently two other competing technologies have been proposed the use of engineered pancreatic β-cell lines (Efrat S, Advanced Drug Delivery Reviews, 1998;33:45-52), and the use of pluripotent embryonal stem cells (Shamblott M J et al., Proc. Natl. Acad. Sci. USA, 1998;95:13726-13731). The former option, while attractive, is associated with significant problems. Not only must the engineered cell be able to produce insulin, but it must respond in a physiologic manner to the prevailing level of glucose- and the glucose sensing mechanism is far from being understood well enough to engineer it into a cell. Many proposed cell lines are also transformed lines, and therefore have a neoplastic potential. With respect to the latter option, having an embryonal stem cell in hand is appealing because of the theoretical possibility of being able to induce differentiation in any direction, including toward the pancreatic β-cell. However, the signals necessary to achieve this milestone remain unknown.

It would be highly desirable to be provided with a platform for the preparation of dedifferentiated intermediate cells derived from post-natal islets of Langerhans or acinar cells, their expansion and the guided induction of islet cell differentiation, leading to insulin-producing cells that can be used for the treatment of diabetes mellitus.

### SUMMARY OF THE INVENTION

One aim of the invention is to provide a platform for the preparation of dedifferentiated intermediate cells derived from post-natal islets of Langerhans or acinar cells, their expansion and the guided induction of islet cell differentiation, leading to insulin-producing cells that can be used for the treatment of diabetes mellitus.

In accordance with one embodiment of the present invention there is provided a medium for preparing duct-like structure cells derived from post-natal islets of Langerhans or acinar cells, which comprises in a physiologically acceptable culture medium an effective amount of:

a) a solid matrix environment for a three-dimensional culture; and

b) a factor for developing, maintaining and expanding said dedifferentiated intermediate cells, said first factor inducing a rise in intracellular cAMP.

In accordance with one embodiment of the present invention there is provided an in vitro method for islet cell expansion, which comprises the steps of:

a) inducing cystic formation in cells cultured in a medium of the present invention, wherein said cells are selected from the group consisting of acinar cells and cells derived from post-natal islets of Langerhans cells to obtain a duct-like structure;

b) expanding cells of said duct-like structure; and

c) inducing islet cell differentiation properties of the expanded cells of said duct-like structure to become insulin-producing cells.

A preferred medium for preparing dedifferentiated cells derived from post-natal islets of Langerhans or acinar cells comprises in a physiologically acceptable culture medium an effective amount of a solid matrix environment for a three-dimensional culture, a matrix protein, and a first and a second factor for developing, maintaining and expanding the dedifferentiated cells.

Preferably the factor may induce a rise in intracellular cAMP, and the factor may be derived from acinar cells. The first factor may comprise one or more of cholera toxin (CT), forskolin, high glucose concentrations, a promoter of cAMP, and EGF.

The matrix protein comprises one or more of laminin, collagen type I and Matrigel™.

Preferably, step c) is effected by retaining cells in the matrix.

Preferably, the culture medium may comprise DMEM/12 supplemented with an effective amount of fetal calf serum, such as 10%. The basal liquid medium may further comprise glucose concentration of at least 11 mM.

In accordance with another embodiment of the present invention there is provided an in vitro method for producing cells with at least bipotentiality, which comprises the steps of:

a) inducing cystic formation in cells cultured in a medium of the present invention, wherein said cells are selected from the group consisting of acinar cells and cells derived from post-natal islets of Langerhans cells from a patient to obtain a duct-like structure; whereby when the duct-like structure cells are introduced in situ in the patient, the cells are expanded and islet cell differentiation properties are induced to become in situ insulin-producing cells.

Preferably, the stem cells are selected from the group consisting of muscle, skin, bone, cartilage, lung, liver, bone marrow and hematopoietic cells.

In accordance with another embodiment of the present invention there is provided a method for the treatment of diabetes mellitus in a patient, which comprises the steps of

a) inducing cystic formation in cells cultured in a medium of the present invention, wherein said cells are selected from the group consisting of acinar cells and cells derived from post-natal islets of Langerhans cells of a patient to obtain a duct-like structure; and

b) introducing the duct-like structure cells in situ in the patient, wherein the cells are expanded in situ and islet cell differentiation properties are induced in situ to become insulin-producing cells.

In accordance with another embodiment of the present invention there is provided a method for the treatment of diabetes mellitus in a patient, which comprises the steps of

a) inducing cystic formation in cells cultured in a medium of the present invention, wherein said cells are selected from the group consisting of acinar cells and cells derived from post-natal islets of Langerhans cells of the patient to obtain a duct-like structure;

b) expanding in vitro the duct-like structure cells;

c) inducing in vitro islet cell differentiation properties of the expanded cells of duct-like structure to become insulin-producing cells; and

d) introducing the cells of step c) in situ in the patient, wherein the cells produce insulin in situ.

For the purpose of the present invention the following terms are defined below.

The expression “post-natal islets of Langerhans” is intended to mean islet cells of any origin, such as human, porcine and canine, among others.

The expression “dedifferentiated cells” is intended to mean cells of any origin which are stem-like cells or cells forming a duct-like structure.

### BRIEF DESCRIPTION OF THE DRAWINGS

FIG. 1 illustrates cell-type conversion from islet to duct-like structure (human tissues), (a) Islet in the pancreas, (b) Islet following isolation and purification, (c) islet in solid matrix beginning to undergo cystic change, (d-f) progressive formation of cystic structure with complete loss of islet morphology.

FIG. 2 illustrates same progression of changes as in FIG. 1. Cells are stained by immunocytochemistry for insulin. (a) Islet in pancreas. (b) Islet after isolation and purification. (c-e) Progressive loss of islet phenotype. (f) High power view of cyst wall composed duct-like epithelial cells. One cell still contains insulin (arrow).

FIG. 3 illustrates same progression of changes as in FIG. 1. Cells stained by immunocytochemistry for glucagon. (a) Islet in pancreas. (b) Islet after isolation and purification. (c-e) Progressive loss of islet phenotype. (f) High power view of cyst wall composed duct-like epithelial cells. One cell still contains glucagon (arrow).

FIGS. 4A-C illustrate demonstration of cell phenotype by CK-19 immunocytochemistry. Upper left panel—cystic structure in solid matrix. All cells stain for CK-19, a marker expressed in ductal epithelial cells in the pancreas. Lower panel-following removal from the solid matrix, and return to suspension culture. A structure exhibiting both epithelial-like and solid components. Upper right panel—only the epithelial-like component retains CK-19 immunoreactivity. The solid component has lost its CK-19 expression, and appears islet-like.

FIGS. 5A-B illustrate upper panel-Ultrastructural appearance of cells composing the cystic structures in solid matrix. Note the microvilli and loss of endosecretory granules. The cells have the appearance of primitive duct-like cells. Lower panel-ultrastructural appearance of cystic structures removed from the solid matrix and placed in suspension culture. Note the decrease in microvilli and the reappearance of endosecretory granules.

FIGS. 6A-B illustrate in situ hybridization for pro-insulin mRNA. Upper panel-cystic structures with virtually no cells containing the message. Lower panel-cystic structures have been removed from the matrix and placed in suspension culture. Note the appearance now, of both solid and cystic structures. The solid structures have an abundant expression of pro-insulin mRNA.

FIG. 7 illustrates insulin release into the culture medium by the structures seen in the lower panel of FIG. 6. Note that there is no demonstrable insulin secreted from the tissue when in the cystic state (far left column). FN-fibronectin; IGF-1-insulin-like growth factor-1; Gluc-glucose.

FIG. 8 illustrates Islets embedded in collagen matrix and maintained in DMEM/F12-CT. Photos from under the inverted microscope (A, C, E) and corresponding histological sections stained for pancytokeratin AE1/AE3 by immunocytochemistry (B, D, F). (A, C, E, ×100; B, D, F, ×200)

FIG. 9 illustrates Islets at an intermediate stage of cystic transformation still contain cells that (A) express the pro-insulin mRNA and that (B) synthesize and store insulin protein. (×400)

FIG. 10A illustrates Intracellular level of cAMP during the time course of islet-cystic transformation. Note the relatively constant level of intracelluar cAMP in islets maintained in CMRL 1066 alone.

FIG. 10B illustrates the integrated amount of cAMP (area under the curve in A) measured at 120 hours. There were no differences observed between islets cultured in DMEM/F12-CT, CMRL-CT and CMRL-forskolin. Note, however, that islets maintained in CMRL alone had significantly less intracellular cAMP.

FIG. 10C illustrates the percentage of islets undergoing cystic transformation increased over the time course of the culture period in the DMEM/F12-CT, CMRL-CT and CMRL-forskolin groups. Islets maintained in CMRL 1066 had a very low level of cystic transformation that remained constant. * p<0.05, ** p<0.01, *** p<0.001

FIG. 11 illustrates the progressive loss of tissue insulin content during the time course of cystic transformation. Note the steep decline in islets maintained in DMEM/F12-CT, CMRL-CT and CMRL-forskolin, which corresponds to the early onset of apoptosis by 16 hours. * p<0.03

FIG. 12 illustrates Apoptotic activity (A) and BrdU labeling index (B) of islets cultured in DMEM/F12-CT and CMRL 1066 over the time course of cystic transformation. Note the shift to the left in the onset of apoptosis in islets in DMEM/F12-CT. *p<0.02; **p<0.01; ***p<0.001.

FIG. 13 illustrates the effect of integrin-binding peptides GRGDSP and GRGESP (A), extracellular matrix proteins laminin and fibronectin (B) and a combination of GRGDSP or GRGESP and laminin (C) on islet-cystic transformation. *p<0.05, **p<0.01. ***p<0.001.

FIG. 14 illustrates the effect of extracellular matrix on islet-cystic transformation in isolated canine islets.

FIG. 15 illustrates the acinar (day 0) to duct differentiation (day 10) according to one embodiment of the present invention.

FIG. 16 illustrates implantation of islet-derived cystic structures plus islets into the submucosal space of the hamster small intestine.

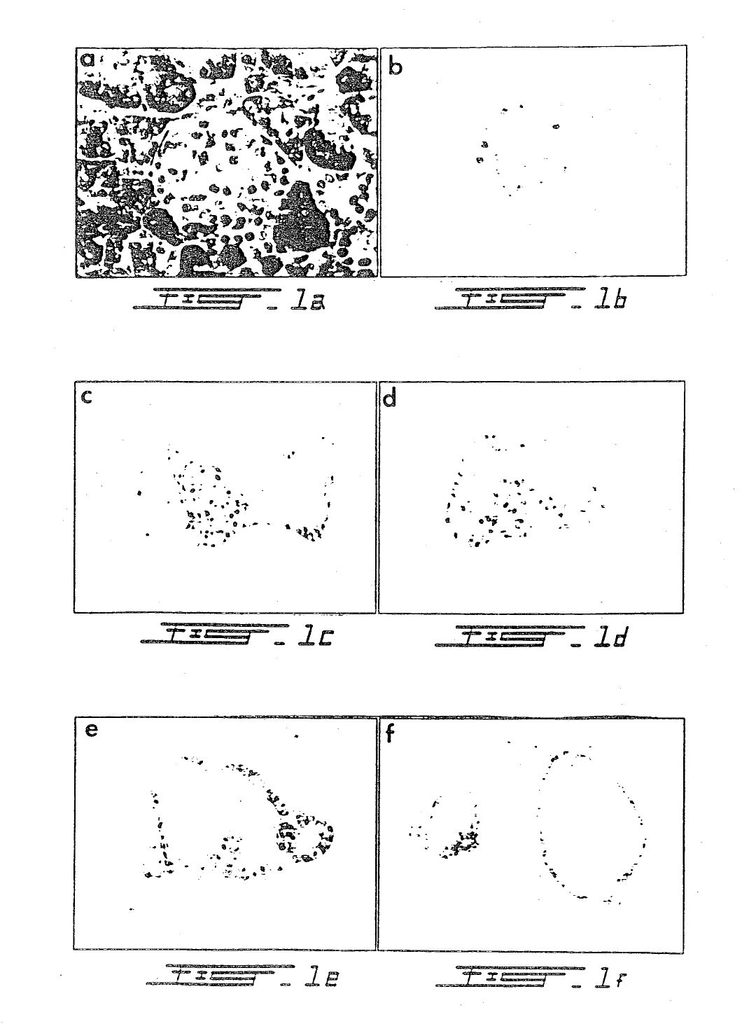

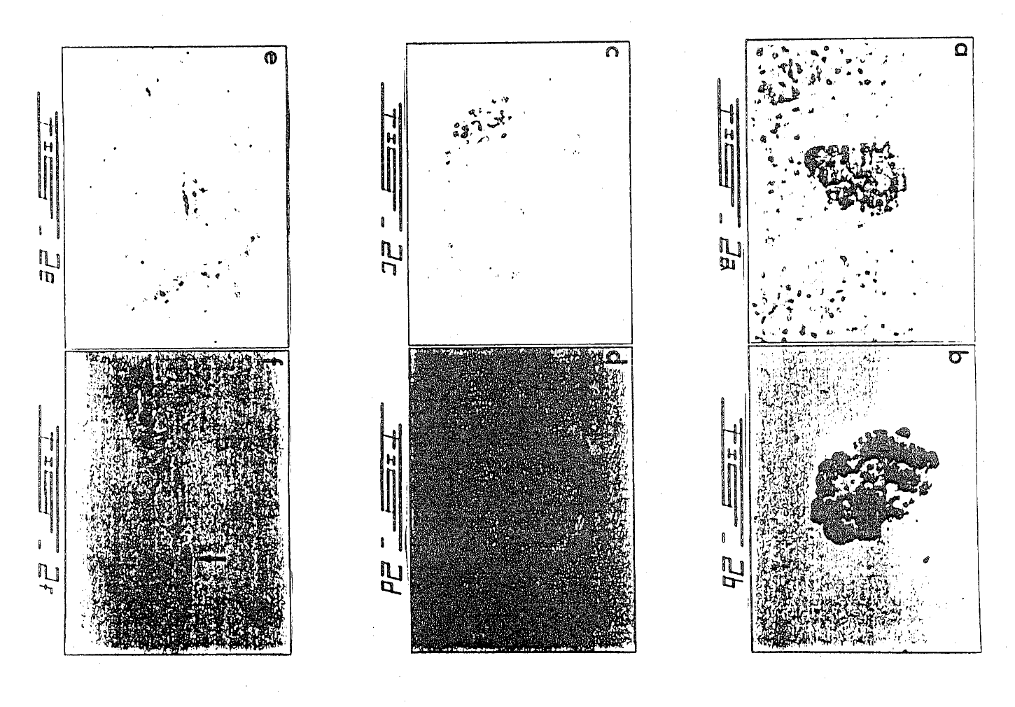

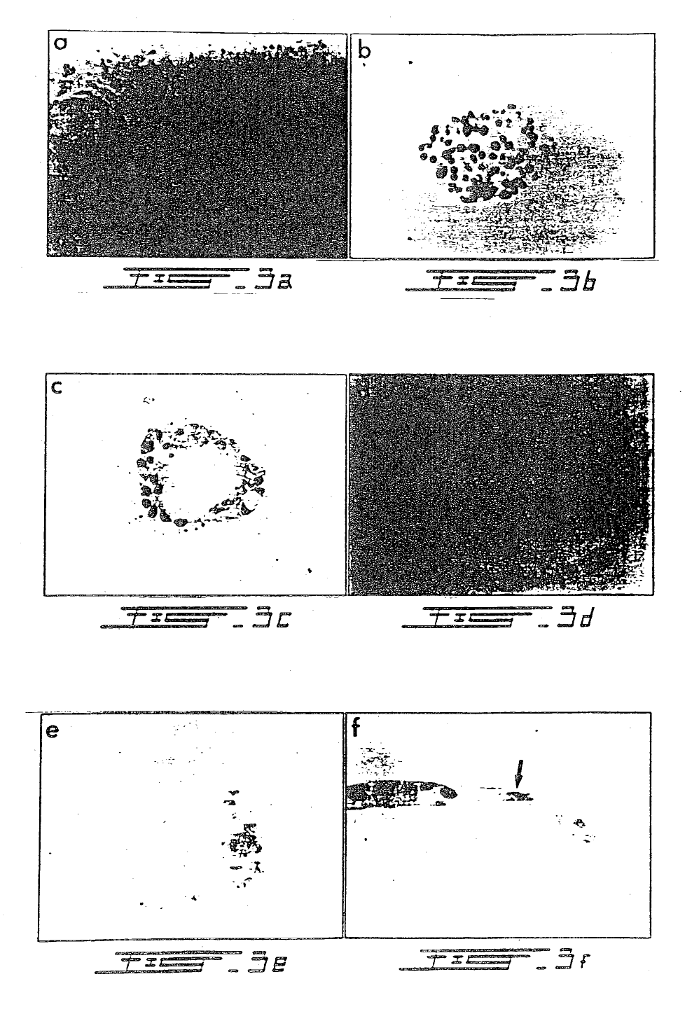

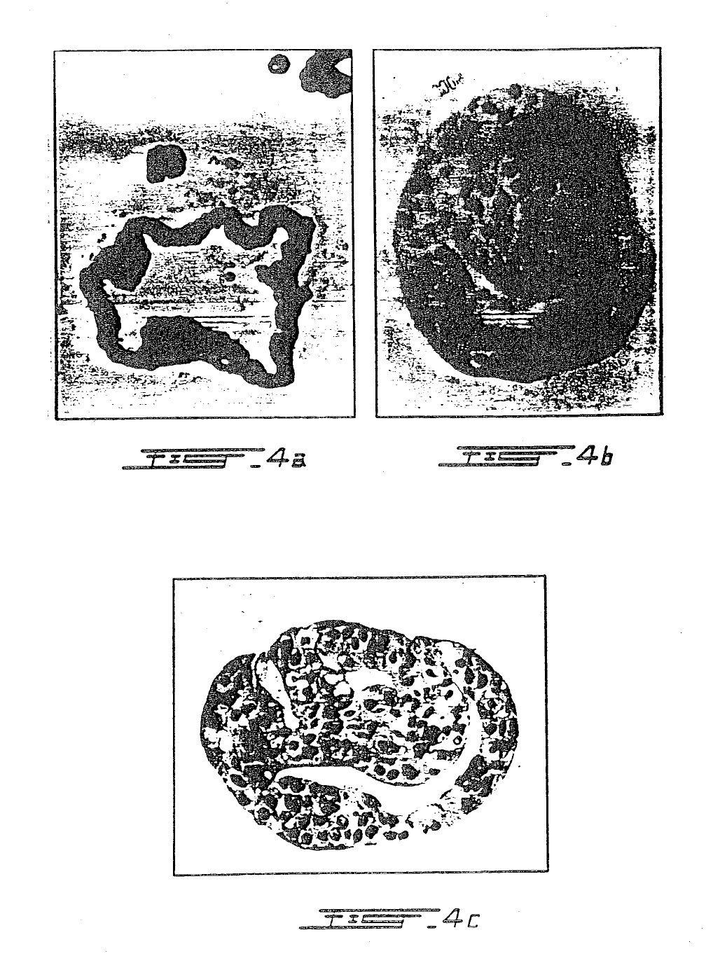

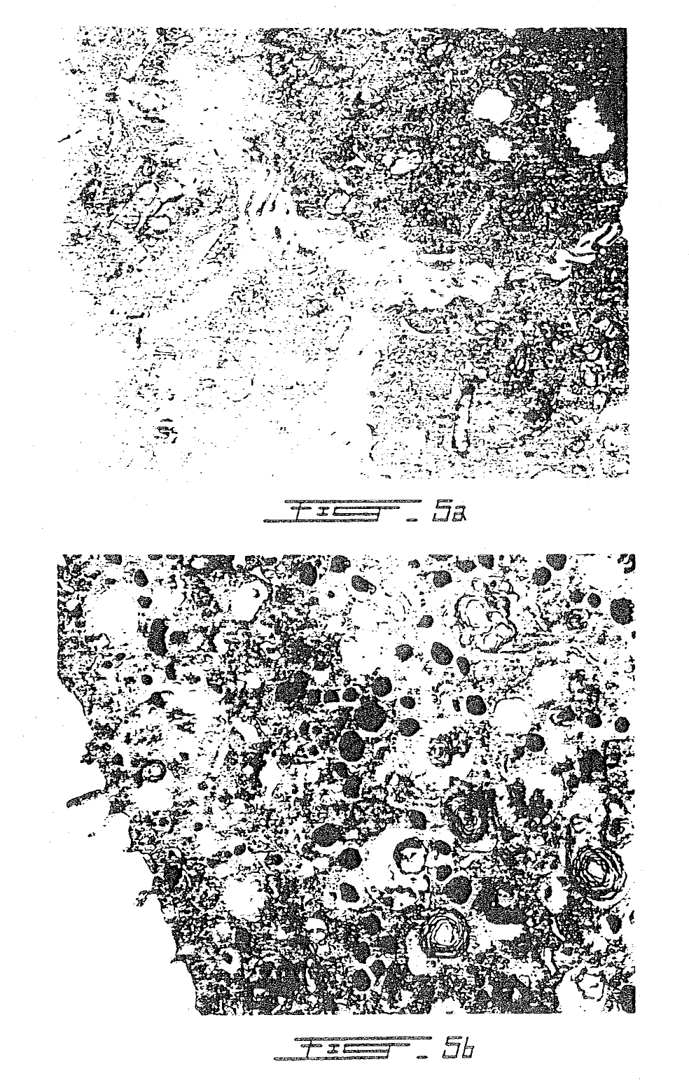

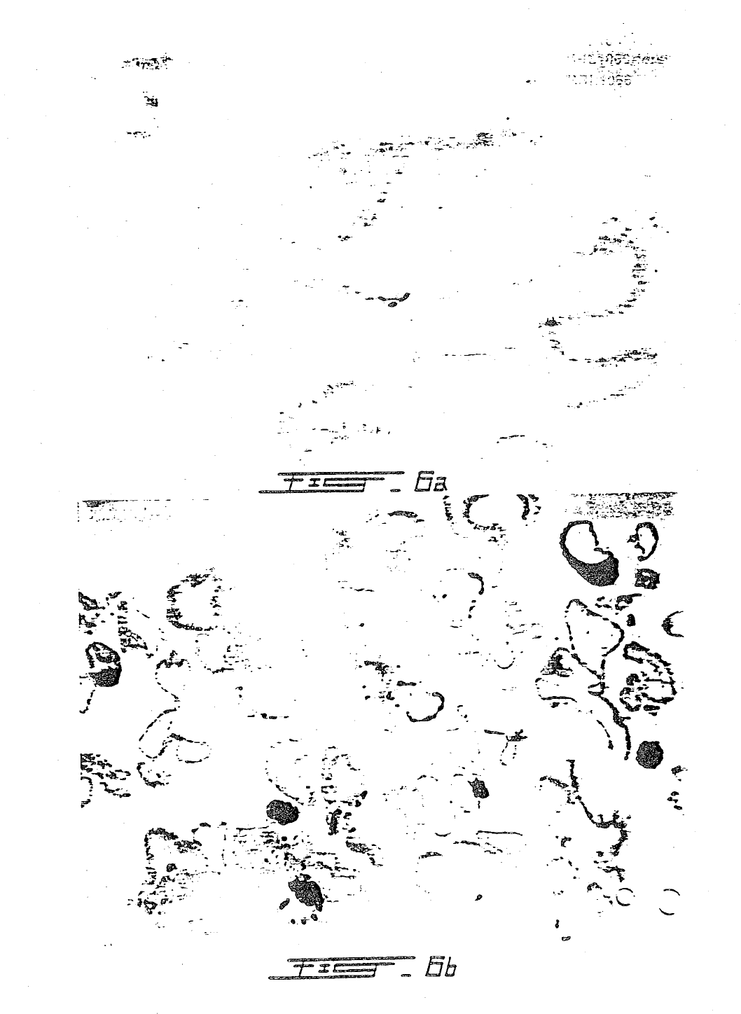

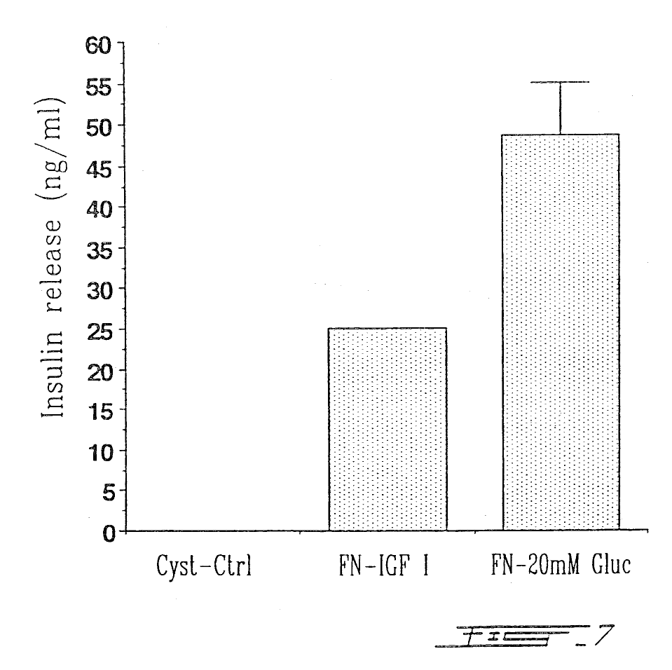

### DETAILED DESCRIPTION OF THE INVENTION

In vivo cell transformation leading to β-cell neogenesis and new islet formation can be understood in the context of established concepts of developmental biology.

Transdifferentiation is a change from one differentiated phenotype to another, involving morphological and functional phenotypic markers (Okada T S., Develop. Growth and Differ. 1986;28:213-321). The best-studied example of this process is the change of amphibian iridial pigment cells to lens fibers, which proceeds through a sequence of cellular dedifferentiation, proliferation and finally redifferentiation (Okada T S, Cell Diff. 1983;13:177-183; Okada T S, Kondoh H, Curr. Top Dev. Biol., 1986;20:1-433; Yamada T, Monogr. Dev. Biol., 1977;13:1-124). Direct transdifferentiation without cell division has also been reported, although it is much less common (Beresford Wash., Cell Differ. Dev., 1990;29:81-93). While transdifferentiation has been thought to be essentially irreversible, i.e. the transdifferentiated cell does not revert back into the cell type from which it arose, this has recently been reported not to be the case (Danto S I et al., Am. J. Respir. Cell Mol. Biol., 1995;12:497-502). Nonetheless, demonstration of transdifferentiation depends on defining in detail the phenotype of the original cells, and on proving that the new cell type is in fact descended from cells that were defined (Okada T S, Develop. Growth and Differ. 1986;28:213-321).

In many instances, transdifferentiation involves a sequence of steps. Early in the process, intermediate cells appear that express neither the phenotype of the original nor the subsequent differentiated cell types, and therefore they have been termed dedifferentiated. The whole process is accompanied by DNA replication and cell proliferation. Dedifferentiated cells are assumed a priori to be capable of forming either the original or a new cell type, and thus are multipotential (Itoh Y, Eguchi G, Cell Differ., 1986;18:173-182; Itoh Y, Eguchi G, Develop. Biology, 1986;115:353-362; Okada T S, Develop. Growth and Differ, 1986;28:213-321).

Stability of the cellular phenotype in adult organisms is probably related to the extracellular milieu, as well as cytoplasmic and nuclear components that interact to control gene expression. The conversion of cell phenotype is likely to be accomplished by selective enhancement of gene expression, which controls the terminal developmental commitment of cells.

The pancreas is composed of several types of endocrine and exocrine cells, each responding to a variety of trophic influences. The ability of these cells to undergo a change in phenotype has been extensively investigated because of the implications for the understanding of pancreatic diseases such as cancer and diabetes mellitus. Transdifferentiation of pancreatic cells was first noted nearly a decade ago. Hepatocyte-like cells, which are normally not present in the pancreas, were observed following the administration of carcinogen (Rao M S et al., Am. J. Pathol., 1983;110:89-94; Scarpelli D G, Rao M S, Proc. Nat. Acad. Sci. USA 1981;78:2577-2581) to hamsters and the feeding of copper-depleted diets to rats (Rao M S, et al., Cell Differ., 1986;18:109-117). Recently, transdifferentiation of isolated acinar cells into duct-like cells has been observed by several groups (Arias A E, Bendayan M, Lab Invest., 1993;69:518-530; Hall P A, Lemoine N R, J. Pathol., 1992;166:97-103; Tsap M S, Duguid W P, Exp. Cell Res., 1987;168:365-375). In view of these observations it is probably germane that during embryonic development, the hepatic and pancreatic anlagen are derived from a common endodermal

Factors which control the growth and functional maturation of the human endocrine pancreas during the fetal and post-natal periods are still poorly understood, although the presence of specific factors in the pancreas has been hypothesized (Pictet R L et al. In: Extracellular Matrix Influences on Gene Expression. Slavkin H C, Greulich R C (eds). Academic Press, New York, 1975, pp.10).

Some information is available on exocrine growth factors. Mesenchymal Factor (MF), has been extracted from particulate fractions of homogenates of midgestational rat or chick embryos. MF affects cell development by interacting at the cell surface of precursor cells (Rutter W J. The development of the endocrine and exocrine pancreas. In: The Pancreas. Fitzgerald P J, Morson A B (eds). Williams and Wilkins, London, 1980, pp.30) and thereby influences the kind of cells that appear during pancreatic development (Githens S. Differentiation and development of the exocrine pancreas in animals. In: Go VLW, et al. (eds). The Exocrine Pancreas: Biology, Pathobiology and Diseases. Raven Press, New York, 1986, pp.21). MF is comprised of at least 2 fundamental components, a heat stable component whose action can be duplicated by cyclic AMP analogs, and another high molecular weight protein component (Rutter W J, In: The Pancreas. Fitzgerald P J, Morson A B (eds). Williams and Wilkins, London, 1980, pp.30). In the presence of MF, cells divide actively and differentiate largely into non-endocrine cells.

Other factors have also been implicated in endocrine maturation. Soluble peptide growth factors (GF) are one group of trophic substances that regulate both cell proliferation and differentiation. These growth factors are multi-functional and may trigger a broad range of cellular responses (Sporn & Roberts, Nature, 332:217-19, 1987). Their actions can be divided into 2 general categories—effects on cell proliferation, which comprises initiation of cell growth, cell division and cell differentiation; and effects on cell function. They differ from the polypeptide hormones in that they act in an autocrine and/or paracrine manner (Goustin A S, Leof E B, et al. Cancer Res., 46:1015-1029, 1986; Underwood L E, et al., Clinics in Endocrinol. & Metabol., 15:59-77,1986). Specifics of their role in the individual processes that comprise growth need to be resolved.

One family of growth factors are the somatomedins. Insulin-like growth factor-I (IGF-I), is synthesized and released in tissue culture by the β-cells of fetal and neonatal rat islets (Hill D J, et al., Diabetes, 36:465-471, 1987; Rabinovitch A, et al., Diabetes, 31:160-164,1982; Romanus J A et al., Diabetes 34:696-792, 1985). IGF-II has been identified in human fetal pancreas (Bryson J M et al., J. Endocrinol., 121:367-373,1989). Both these factors enhance neonatal β-cell replication in vitro when added to the culture medium (Hill D J, et al., Diabetes, 36:465-471, 1987; Rabinovitch A, et al., Diabetes, 31:160-164, 1982). Therefore the IGF's may be important mediators of β-cell replication in fetal and neonatal rat islets but may not do so in post-natal development (Billestrup N, Martin J M, Endocrinol., 116:1175-81,1985; Rabinovitch A, et al., Diabetes, 32:307-12, 1983; Swenne I, Hill D J, Diabetologia 32:191-197, 1989; Swenne I, Endocrinology, 122:214-218, 1988; Whittaker P G, et al, Diabetologia, 18:323-328, 1980). Furthermore, Platelet-derived growth factor (PDGF) also stimulates fetal islet cell replication and its effect does not require increased production of IGF-I (Swenne I, Endocrinology, 122:214-218, 1988). Moreover, the effect of growth hormone on the replication of rat fetal B-cells appears to be largely independent of IGF-I (Romanus J A et al., Diabetes 34:696-792, 1985; Swenne I, Hill D J, Diabetologia 32:191-197, 1989). In the adult pancreas, IGF-I mRNA is localized to the D-cell. But IGF-I is also found on cell membranes of β- and A-cells, and in scattered duct cells, but not in acinar or vascular endothelial cells (Hansson H-A et al., Acta Physiol. Scand. 132:569-576, 1988; Hansson H-A et al., Cell Tissue Res., 255:467-474, 1989). This is in contradistinction to one report (Smith F et al, Diabetes, 39 (suppl 1):66A, 1990), wherein IGF-I expression was identified in ductular and vascular endothelial cells, and appeared in regenerating endocrine cells after partial pancreatectomy. It has not been shown that IGF's will stimulate growth of adult duct cells or islets. Nor do the IGF's stimulate growth of the exocrine pancreas (Mossner J et al., Gut 28:51-55, 1987). It is apparent therefore, that the role of IGF-I, especially in the adult pancreas, is far from certain.

Fibroblast growth factor (FGF) has been found to initiate transdifferentiation of the retinal pigment epithelium to neural retinal tissues in chick embryo in vivo and in vitro (Hyuga M et al., Int. J. Dev. Biol. 1993;37:319-326; Park C M et al., Dev. Biol. 1991;148:322-333; Pittack C et al., Development 1991;113:577-588). Transforming growth factor-beta (TGF-β) has been demonstrated to induce transdifferentiation of mouse mammary epithelial cells to fibroblast cells [20]. Similarly, epithelial growth factor (EGF) and cholera toxin were used to enhance duct epithelial cyst formation from among pancreatic acinar cell fragments (Yuan S et al., In vitro Cell Dev. Biol., 1995;31:77-80).

The search for the factors mediating cell differentiation and survival must include both the cell and its microenvironment (Bissell M J et al., J. Theor. Biol., 1982; 99:31), as a cell's behavior is controlled by other cells as well as by the extracellular matrix (ECM) (Stoker A W et al. Curr. Opin. Cell. Biol., 1990;2:864). ECM is a dynamic complex of molecules serving as a scaffold for parenchymal and nonparenchymal cells. Its importance in pancreatic development is highlighted by the role of fetal mesenchyme in epithelial cell cytodifferentiation (Bencosme S A, Am. J. Pathol. 1955; 31: 1149; Gepts W, de Mey J. Diabetes 1978; 27(suppl. 1): 251; Gepts W, Lacompte P M. Am. J. Med., 1981; 70: 105; Gepts W. Diabetes 1965; 14: 619; Githens S. In: Go VLW, et al. (eds) The Exocrine Pancreas: Biology, Pathobiology and Disease. (New York: Raven Press, 1986) p. 21). ECM is found in two forms—interstitial matrix and basement membrane (BM). BM is a macromolecular complex of different glycoproteins, collagens, and proteoglycans. In the pancreas, the BM contains laminin, fibronectin, collagen types IV and V, as well as heparan sulphate proteoglycans (Ingber D. In: Go VLW, et al (eds) The Pancreas: Biology, Pathobiology and Disease (New York: Raven Press, 1993) p. 369). The specific role of these molecules in the pancreas has yet to be determined.

ECM has profound effects on differentiation. Mature epithelia that normally never express mesenchymal genes, can be induced to do so by suspension in collagen gels in vitro (Hay E D. Curr. Opin. in Cell. Biol. 1993; 5:1029). For example, mammary epithelial cells flatten and lose their differentiated phenotype when attached to plastic dishes or adherent collagen gels (Emerman J T, Pitelka D R. In vitro 1977; 13:316). The same cells round, polarize, secrete milk proteins, and accumulate a continuous BM when the gel is allowed to contract (Emerman J T, Pitelka D R. In vitro, 1977; 13:316). Thus different degrees of retention or re-formation of BM are crucial for cell survival and the maintenance of the normal epithelial phenotype (Hay E D. Curr. Opin. in Cell. Biol. 1993; 5:1029).

During times of tissue proliferation, and in the presence of the appropriate growth factors, cells are transiently released from ECM-determined survival constraints. It is now becoming clear that there are two components of the cellular response to ECM interactions—one physical, involving shape changes and cytoskeletal organization; the other biochemical, involving integrin clustering and increased protein tyrosine phosphorylation (Ingber Del. Proc. Natl. Acad. Sci. USA, 1990;87:3579; Roskelley C D et al., Proc. Natl. Acad. Sci. USA, 1994;91:12378).

In addition to its known regulatory role in cellular growth and differentiation, ECM has more recently been recognized as a regulator of cell survival (Bates R C, Lincz L F, Burns G F, Cancer and Metastasis Rev., 1995;14:191). Disruption of the cell-matrix relationship leads to apoptosis (Frisch S M, Francis H. J. Cell. Biol., 1994;124:619; Schwartz S M, Bennett M R, Am. J. Path., 1995;147:229), a morphological series of events (Kerr J F K et al., Br. J. Cancer, 1972;26:239), indicating a process of active cellular self destruction.

In accordance with one embodiment of the present invention, the platform technology is based on a combination of the foregoing observations, incorporating in a basal feeding medium the following components that are necessary and sufficient for the preparation of dedifferentiated intermediate cells from adult pancreatic islets of Langerhans:

1. a solid matrix permitting “three dimensional” culture;

2. the presence of matrix proteins including but not limited to collagen type I and laminin; and

3. the growth factor EGF and promoters of cAMP, including but not limited to cholera toxin and forskolin.

The preferred feeding medium is DMEM/F12 with 10% fetal calf serum. In addition, the starting tissue must be freshly isolated and cultured without absolute purification.

The use of a matrix protein-containing solid gel is an important part of the culture system, because extracellular matrix may promote the process of transdifferentiation. This point is highlighted by isolated pancreatic acinar cells, which transdifferentiate to duct-like structures when entrapped in Matrigel basement membrane (Arias A E, Bendayan M, Lab Invest., 1993;69:518-530), or by retinal pigmented epithelial cells, which transdifferentiate into neurons when plated on laminin-containing substrates (Reh T A et al., Nature 1987;330:68-71). Most recently, Gittes et al. demonstrated, using 11-day embryonic mouse pancreas, that the default path for growth of embryonic pancreatic epithelium is to form islets (Gittes G K et al., Development 1996; 122:439-447). In the presence of basement membrane constituents, however, the pancreatic anlage epithelium appears to programmed to form ducts. This finding again emphasizes the interrelationship between ducts and islets and highlights the important role of the extracellular matrix.

This completes stage 1 (the production of dedifferentiated intermediate cells) of the process. During the initial 96 h of culture, islets undergo a cystic transformation associated with (Arias A E, Bendayan M, Lab. Invest., 1993;69:518-530) a progressive loss of insulin gene expression, (2) a loss of immunoreactivity for insulin protein, and (3) the appearance of CKA 19, a marker for ductal cells. After transformation is complete, the cells have the ultrastructural appearance of primitive duct-like cells. Cyst enlargement after the initial 96 h is associated, at least in part, with a tremendous increase in cell replication. These findings are consistent with the transdifferentiation of an islet cell to a ductal cell (Yuan et al., Differentiation, 1996; 61:67-75, which showed that isolated islets embedded in a collagen type I gel in the presence of a defined medium undergo cystic transformation within 96 hours).

Stage 2—the generation of functioning β-cells, requires a complete change of the culture conditions. The cells are moved from the digested matrix and resuspended in a basal liquid medium such as CMRL 1066 supplemented with 10% fetal calf serum, with the addition of soluble matrix proteins and growth factors that include, but are not limited to, fibronectin (10-20 ng/ml), IGF-1 (100 ng/ml), IGF-2 (100 ng), insulin (10-100 μg/ml), NGF (10-100 ng/ml). In addition, the glucose concentration must be increased to above 11 mM. Additional culture additives may include specific inhibitors of known intracellular signaling pathways of apoptosis, including, but not limited to a specific inhibitor of p38.

Evidence for the return to an islet cell phenotype includes: (1) the re-appearance of solid spherical structures; (2) loss of CK-19 expression; (3) the demonstration of endosecretory granules on electron microscopy; (4) the re-appearance of pro-insulin mRNA on in situ hybridization; (5) the return of a basal release of insulin into the culture medium.

The present invention will be more readily understood by referring to the following examples which are given to illustrate the invention rather than to limit its scope.

### EXAMPLE I

### Preparation of a Basal Feeding Medium

The purpose of this study was to elucidate the mechanisms involved in the process of transdifferentiation.

Canine islets were isolated using Canine Liberase™ and purified on a Euroficoll gradient in a Cobe 2991 Cell Separator. Freshly isolated islets were embedded in collagen type I gel for up to 120 hr and cultured in (i) DMEM/F12 plus cholera toxin (CT); (ii) CMRL 1066 supplemented with CT; (iii) CMRL 1066 supplemented with forskolin, and (iv) CMRL 1066 alone. At 16 hr, intracellular levels of cAMP (fmol/10³ islets), determined by ELISA, were increased in Groups (i)-(iii) (642±17, 338±48, 1128±221) compared to Group iv (106±19, p<0.01). Total intracellular CAMP at 120 hr (integrated area under the curve) coincided with the % of islets undergoing transdifferentiation (63±2, 48±2, 35±3, 8±1), as determined by routine histology, immunocytochemistry for cytokeratin AE1/AE3, and by a loss of pro-insulin gene expression on in situ hybridization.

To evaluate the role of matrix proteins and the 3-D environment, islets were embedded in collagen type I, Matrigel™ and agarose gel and cultured in DMEM/F12 plus CT. Islets in collagen type I and Matrigel™ demonstrated a high rate of cystic transformation (63±2% and 71±4% respectively), compared to those in agarose (0±0%, p<0.001). In addition, islet cell transdifferentiation was partially blocked by prior incubation of freshly isolated islets with an RGD motif-presenting synthetic peptide.

In conclusion, these studies confirm the potential of freshly isolated islets to undergo epithelial cell transdifferentiation. Elevated levels of intracellular cAMP and matrix proteins presented in a 3-dimensional construct are necessary for this transformation to be induced. The precise nature of the resulting epithelial cells, and the reversibility of the process remain to be determined.

### EXAMPLE II

### Factors Mediating the Transformation of Islets of Langerhans to Duct Epithelial-Like Structures

Materials and Methods

Islet Isolation and Purification

Pancreata from six mongrel dogs of both sexes (body weight 25-30 kg) were resected under general anesthesia in accordance with Canadian Council for Animal Care guidelines (Wang R N, Rosenberg L (1999) J Endocrology 163 181-190). Prior to removal, the pancreatic ducts were cannulated to permit intraductal infusion with Liberase CI® (1.25 mg/ml) (Boehringer Mannheim, Indianapolis, Ind., USA) according to established protocols (Horaguchi A, Merrell R C (1981) Diabetes 30 455-461; Ricordi C (1992) Pancreatic islet cell transplantation. pp99-112. Ed Ricordi C. Austin: R. G. Landes Co.). Purification was achieved by density gradient separation in a three-step EuroFicoll gradient using a COBE 2991 Cell Processor (COBE BCT, Denver, Colo., USA) (London N J M et al. (1992) Pancreatic islet cell transplantation. pp113-123. Ed Ricordi C. Austin: R. G. Landes Co.). The final preparation consisted of 95% dithizone-positive structures with diameters ranging from 50 to 500 μm.

Experimental Design

To evaluate the role of intracellular cAMP, freshly isolated islets were embedded in type 1 collagen gel (Wang R N, Rosenberg L (1999) J Endocrology 163 181-190) and cultured in: (i) DMEM/F12 (GIBCO, Burlington, ON, CANADA) supplemented with 10% FBS, EGF (100 ng/ml) (Sigma, St. Louis, St. Louis, Mo., USA) and cholera toxin (100 ng/ml) (Sigma, St. Louis, Mo., USA); (ii) CMRL1066 (GIBCO) supplemented with 10% FBS and cholera toxin (100 ng/ml) and 16.5 mM D-glucose; (iii) CMRL1066 supplemented with 10% FBS and 2 μM forskolin (Sigma, St. Louis, Mo., USA), and (iv) CMRL1066 supplemented with 10% FBS. Approximately 3000 islets per group per time point were used. Islets were cultured in 95% air/5% CO₂ at 37° C., and the medium was changed on alternate days. Representative islets from each group were examined after isolation (0 hour), and then on hours of 1, 16, 36, 72 and 120 using the following investigations.

The following series of experiments were conducted to evaluate the role of cell-matrix interactions in the process of cystic transformation. First, to determine whether the process required a solid gel environment, islets were cultured in suspension in DMEM/F12 with 10% FBS plus CT and EGF. To determine whether a solid gel environment and extracellular matrix proteins were independent requirements, islets were embedded in 1.5% agarose gel and maintained in DMEM/F12 with 10% FBS plus CT and EGF. Alternatively, islets were cultured in suspension with in DMEM/F12 with 10% FBS plus CT and EGF in the presence of soluble Laminin (50 μg/ml) or Fibronectin (50 μg/ml) (Peninsula Laboratories). To determine whether the process was, at least in part, integrin-mediated, islets were pre-incubated at 37° C. for 60 min either in the presence of the RGD-motif containing GRGDSP peptide or the control peptide GRGESP (400 μg/ml) (Peninsula Laboratories). Finally, to determine whether cystic transformation was dependent on type 1 collagen alone, islets were also embedded in Matrigel® (Peninsula Laboratories, Belmont, Calif., USA).

Morphological Analysis

Immunocytochemistry

Tissue was fixed in 4% paraformaldehyde (PFA) and embedded in 2% agarose following a standard protocol of dehydration and paraffin embedding Wang R N, Rosenberg L (1999) J Endocrology 163 181-190). A set of six serial sections (thickness 4 μm) was cut from each paraffin block.

Consecutive sections were processed for routine histology and immunostained for pancreatic hormones (insulin, glucagon and somatostatin, Biogenex, San Ramon, Calif., USA) and the pan-cytokeratin cocktail AE1/AE3 (Dako, Carpinteria, Calif., USA), using the AB complex method (streptavidin-biotin horseradish peroxidase; Dako), as described previously (Wang R N et al. (1994) Diabetologia 37 1088-1096). For cytokeratin AE1/AE3, sections were pretreated with 0.1% trypsin. The sections were incubated overnight at 4° C. with the appropriate primary antibodies. Negative controls involved the omission of the primary antibodies.

In situ Hybridization

In situ hybridization for human proinsulin mRNA (Novocastra, Burlington, ON, Canada) was performed on consecutive sections of freshly isolated islets and epithelial cystic structures at 120 h. The sections were hybridized with a fluorescein labelled oligonucleotide cocktail solution for 2 h at 37° C. Slides were then incubated with rabbit Fab anti-FITC conjugated to alkaline phosphatase antibody (diluted 1:200) for 30 min at room temperature. The reaction product was visualised by an enzyme-catalysed colour reaction using a nitro blue tetrazolium and 5′-bromo-4-chloro-3-indolyl-phosphate kit (Wang R N, Rosenberg L (1999) J Endocrology 163 181-190, Wang R N et al. (1994) Diabetologia 37 1088-1096).

Analysis of Intracellular cAMP Level

Cells were harvested from the collagen gel and washed in 1 mM cold PBS. Following addition of 200 μl of lysis buffer, each sample was sonicated for 30 s, then incubated for 5 min at room temperature. 1001 of cell lysate was transferred to donkey anti-rabbit Ig coated plate. The intracellular cAMP content of non-acetylated samples was measured using a commercially available cAMP enzyme-linked immunoassay kit (assay range 12.5-3200 fmol/well, Ameraham, Little Chalfont, U.K.). The data are expressed as fmol per 10³ islets.

Insulin Content Assay

Cellular insulin content was measured using a solid-phase radioimmunoassay (Immunocorp, Montreal, Quebec, Canada) (Wang R N, Rosenberg L (1999) J Endocrology 163 181-190) with a sensitivity of 26.7 pmol/l (0.15 ng/ml), an inter-assay variability of <5%, and an accuracy of 100%. The kit uses anti-human antibodies that cross-react with canine insulin. Obtained values were corrected for variations in cell number by measuring DNA content using a fluorometric DNA assay (Yuan S et al. (1996) Differentiation 61, 67-75). The data are expressed as μg per μg DNA.

Cell Death And Proliferation

Cells cultured in DMEM/F12-CT and CMRL1066 were harvested from the gel using collagenase XI (0.25 mg/ml) (Sigma, Montreal, Que.) and processed for a specific programmed cell death ELISA, that detects histone-associated DNA fragments in the cell cytoplasm-a hallmark of the apoptotic process (Roche Molecular, Montreal, Que.) (Paraskevas S et al. (2000) Ann. Surgery in press). Cells were incubated in lysis buffer for 30 min, and the supernatant containing cytoplasmic oligonucleosomes was measured at an absorbance of 405 nm. Variations in sample size were corrected by measuring total sample DNA content (Yuan S et al. (1996) Differentiation 61, 67-75).

To evaluate cell proliferation, cells cultured in DMEM/F12-CT and CMRL1066 were pre-incubated with 10 μM 5-bromo-2′-deoxyuridine (BrdU, Sigma) for 1 h at 37° C. Harvested cells was fixed in 4% PFA as described above. Immunostaining for BrdU was performed using the AB complex method. The sections were pretreated with 0.1% trypsin and 2N HCl denatured DNA. A monoclonal anti-BrdU antibody was used at 1:500 dilution (Sigma). To calculate a BrdU labeling index, the number of cells positive for the BrdU reaction was determined and expressed as a percentage of the total number of cells counted. For each experimental group and time point, at least 500 cells were counted per section.

Statistic Analysis

Data obtained from the six different islet isolations are expressed as mean±SEM. The difference between groups was evaluated by one-way analysis of variance.

RESULTS

Morphological Changes

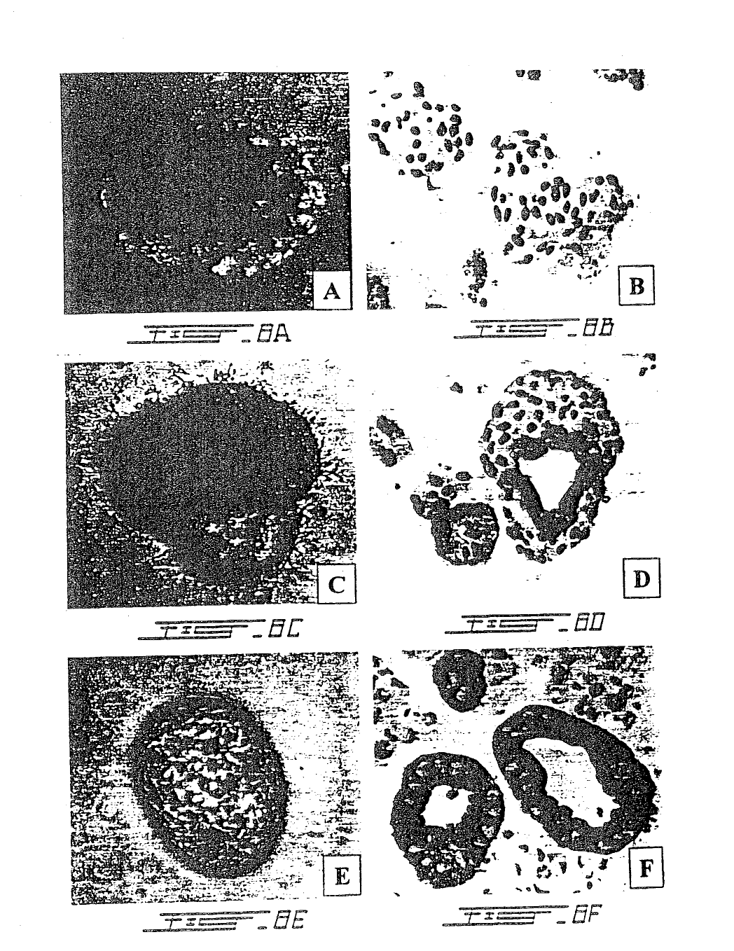

Under the inverted microscope, freshly isolated islets appeared as solid spheroids. At this time, cytokeratin-positive cells were not demonstrated (FIGS. 8A-B).

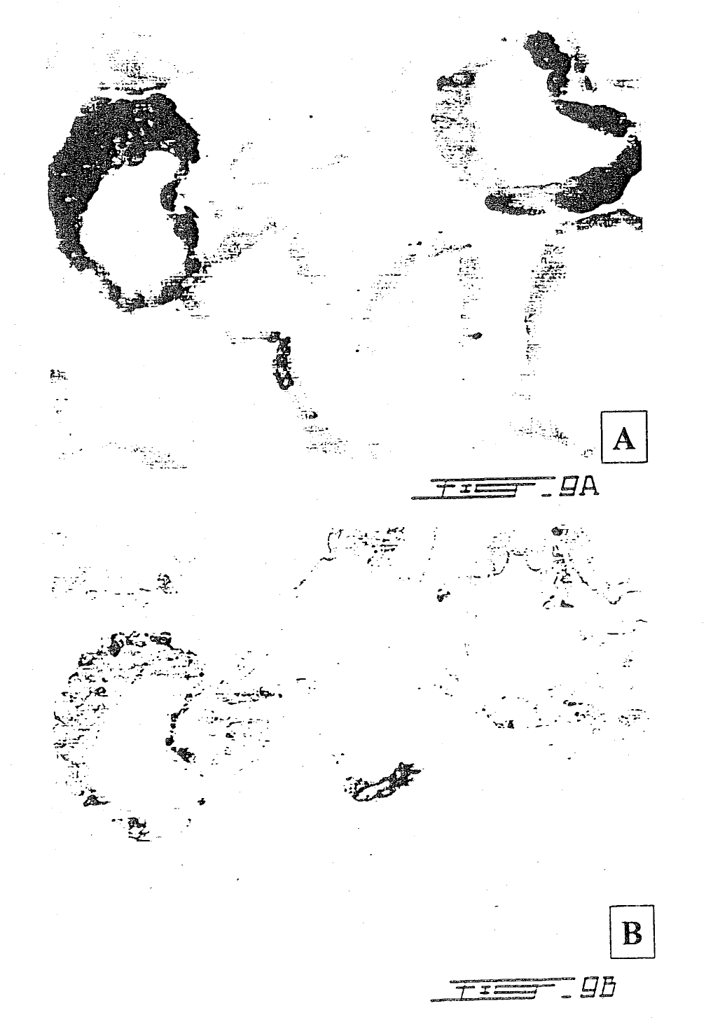

For islets embedded in type 1 collagen and cultured in DMEM/F12 plus CT, CMRL 1066 plus CT or CMRL 1066 plus forskolin, duct epithelial differentiation was first observed coincident with a loss of cells in the islet periphery, at approximately 16 hours. At this time, cells lining the cystic spaces were cytokeratin-positive (FIGS. 8C-D). Fully developed epithelial structures were present in culture by 72 hours (FIGS. 8E-F). Islets cultured in CMRL 1066 alone maintained a solid spheroid appearance for the duration of the study and did not undergo epithelial transformation. Immunocytochemical staining did not demonstrate co-localization of cytokeratin and islet cell hormones. This is in keeping with the observation in the intact pancreas, that cytokeratin staining was only seen on duct epithelial cells. Pro-insulin gene expression and insulin protein were progressively lost during the period of duct epithelial differentiation (FIG. 9) Intracellular cAMP

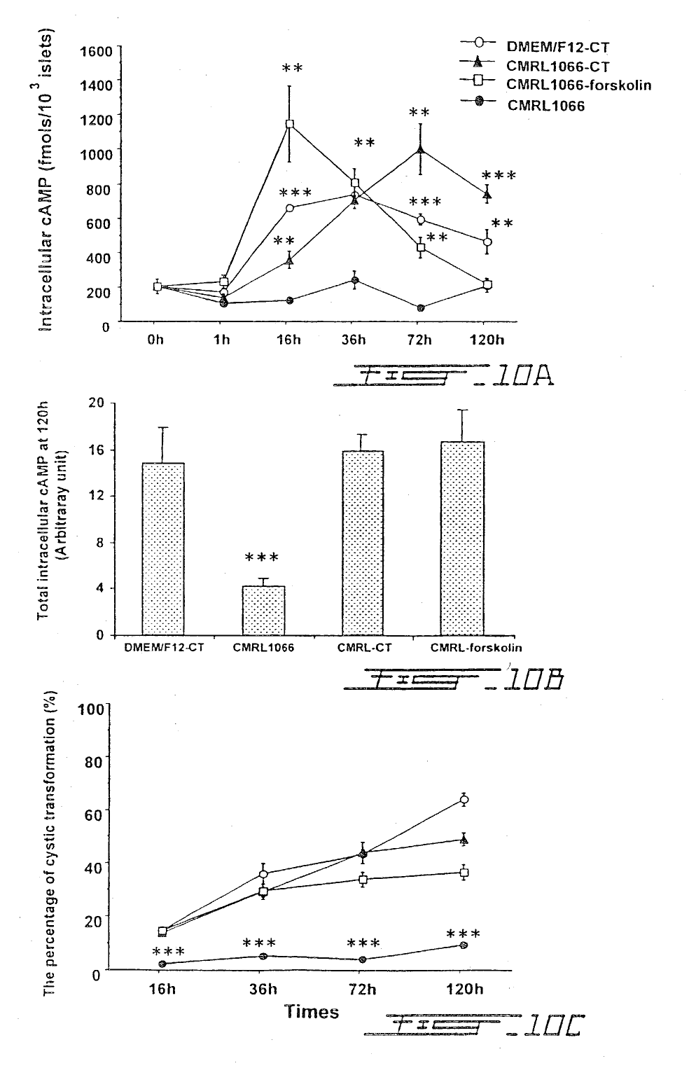

After 1 hour, intracellular levels of cAMP of islets maintained in DMEM/F12-CT, CMRL1066-CT and CMRL1066-forskolin were significantly elevated compared to freshly isolated islets or to islets maintained in CMRL 1066 alone (FIG. 10A). In fact the intracellular level of cAMP of islets cultured in CMRL 1066 alone did not increase at all during the time course of the study. The total intracellular cAMP measured over 120 hr (integrated area under the curve) was similar for islets cultured in DMEM/F12-CT, CMRL 1066-CT and CMRL 1066-forskolin (15±3, 16±2, 17±3 respectively), although the most sustained elevation of cAMP was in the DMEM/F12-CT islets, which were exposed to both EGF and CT. In comparison, islets cultured in CMRL 1066 alone had the lowest level of total intracellular cAMP (4±1, p<0.001) (FIG. 10B), and this translated into the lowest level of islet-duct transformation (FIG. 10C).

Intracellular Insulin Content

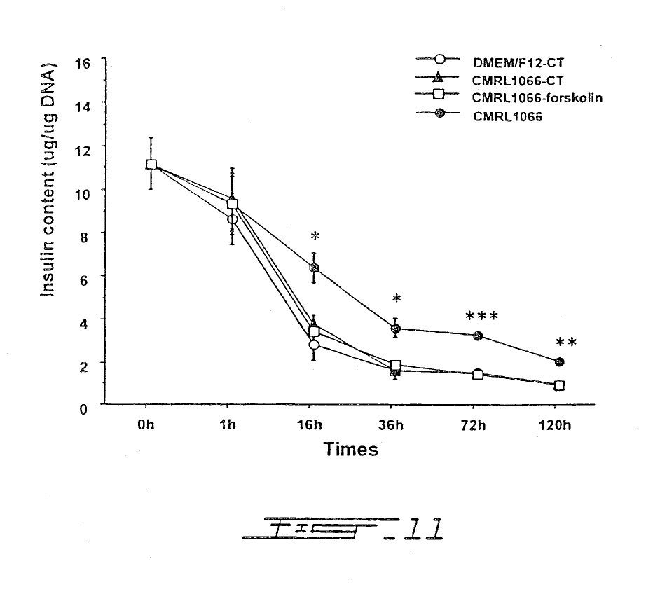

The cellular content of insulin (FIG. 11) was highest in freshly isolated islets (11±2 μg/μg DNA). After 16 hours in culture, the insulin content of cells cultured in DMEM/F12-CT, CMRL1066-CT and CMRL1066-forskolin declined dramatically, falling to 7% of the initial value by 120 hours. Islets cultured in CMRL1066 alone did not undergo epithelial transformation, and maintained a higher level of intracellular insulin compared to the other three groups (p<0.03, FIG. 11).

Analysis Of Cell Death And Proliferation

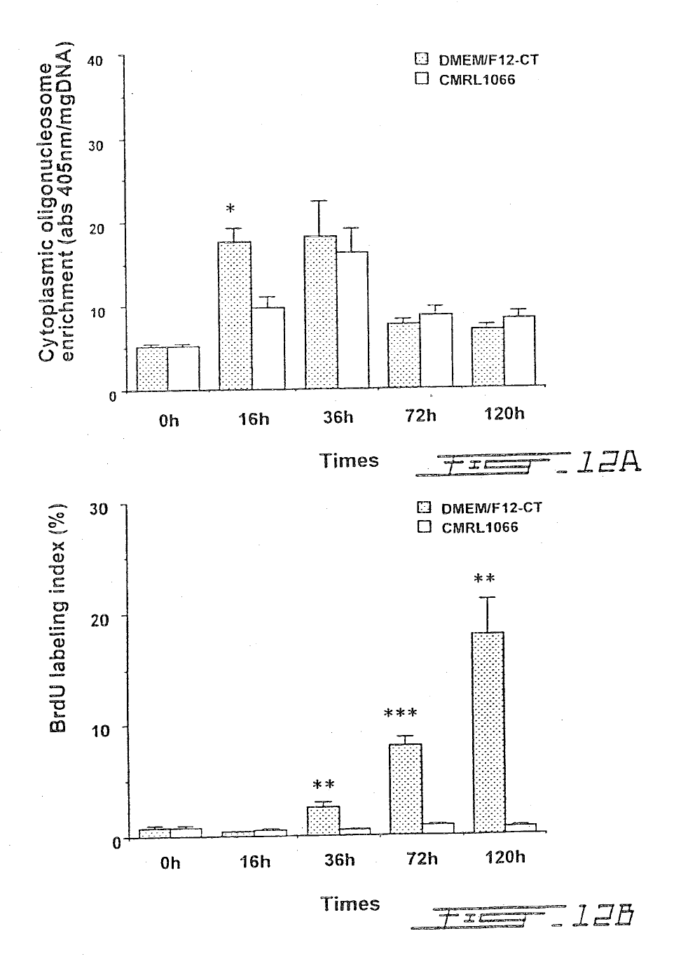

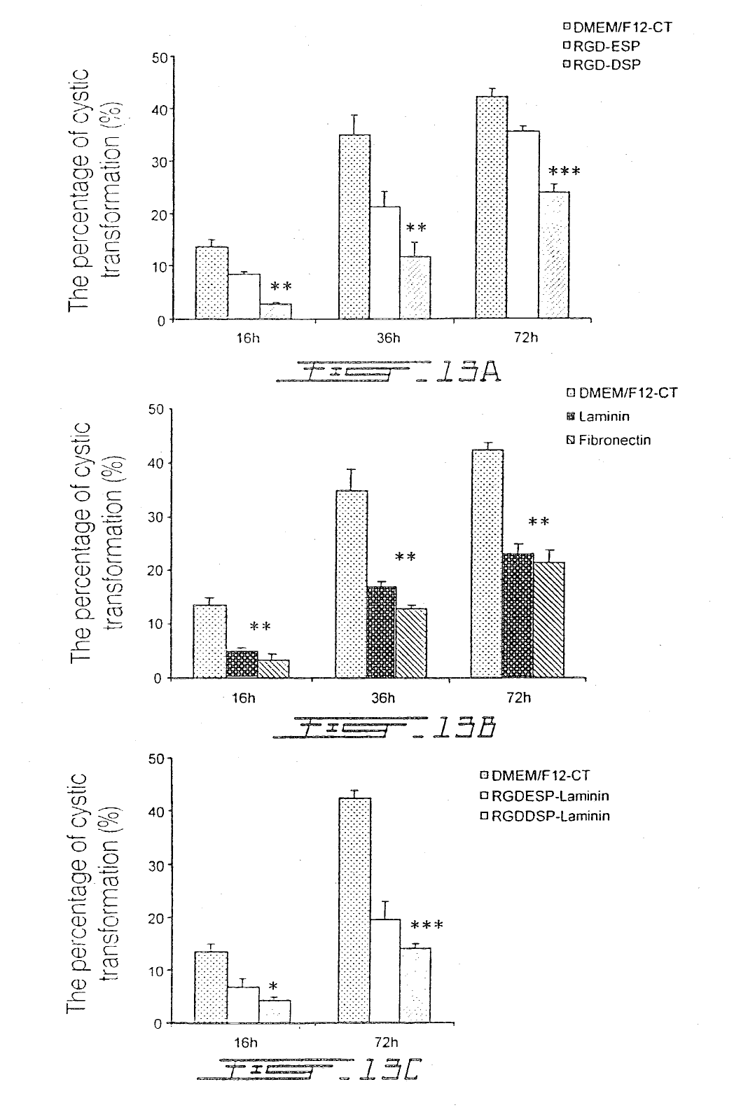

To determine whether cell loss during cystic transformation was due, at least in part, to programmed cell death, we used a specific cell death ELISA. At 16 hours, cytoplasmic oligonucleosome enrichment was significantly higher in islets cultured with DMEM/F12-CT compared to islets cultured in CMRL1066 alone (p<0.02, FIG. 12A). After 36 hours, there was no difference between the groups. Looking at the data as a whole (FIG. 12A), it appears that a wave of apoptosis occurred in both groups of islets, but that the time course of cell death was shifted to the left for islets undergoing cystic transformation in DMEM/F12-CT.

To assess proliferation, cells were labeled with BrdU. Following isolation, the BrdU cell labeling index of islets cultured in DMEM/F12-CT was 0.8% —identical to that of islets cultured in CMRL 1066 alone. After 36 hours, however, a wave of cell proliferation ensued in the DMEM/F12-CT group, with the labeling index reaching 18% at 120 hours (FIG. 12B). In comparison, the labeling index for islets in CMRL 1066 remained essentially unchanged throughout the study period (p<0.01).

The Role Integrin-ECM Interactions

To determine whether elevation of intracelluar cAMP was sufficient to induce duct epithelial differentiation, islets were maintained in suspension culture in DMEM/F12-CT and not embedded in collagen gel. Under these conditions, epithelial transformation did not occur. This suggested that an increase in intracellular cAMP was a necessary but not sufficient requirement for transformation, and that the matrix must also play an important role in the process.

To determine whether it was the solid gel environment or the presence of extracellular matrix proteins alone that was necessary, islets were embedded in agarose gel, type 1 collagen gel or Matrigel®. Only islets embedded in the latter two gels underwent cystic transformation (Table 1). Furthermore, islets maintained in suspension in DMEM/F12-CT supplemented with either soluble laminin or fibronectin, failed to undergo ductal transformation. These experiments indicated that the process of transformation required the presence of ECM proteins presented in a solid gel environment.

TABLE 1
The effect of extracellular matrix on islet-cystic
transformation in isolated canine islets

| | | Collagen | | Soluble |
|---|---|---|---|---|
| Times | Matrigel | I | Agarose | laminin/fibronectin^{a} |
| 16 h | 19 ± 4.7 | 14 ± 1.4 | — | — |
| 36 h | 49 ± 3.7 | 35 ± 3.9 | — | — |
| 72 h | 60 ± 3.7 | 42 ± 1.6 | — | — |
| 120 h | 71 ± 4.5 | 63 ± 2.4 | — | — |

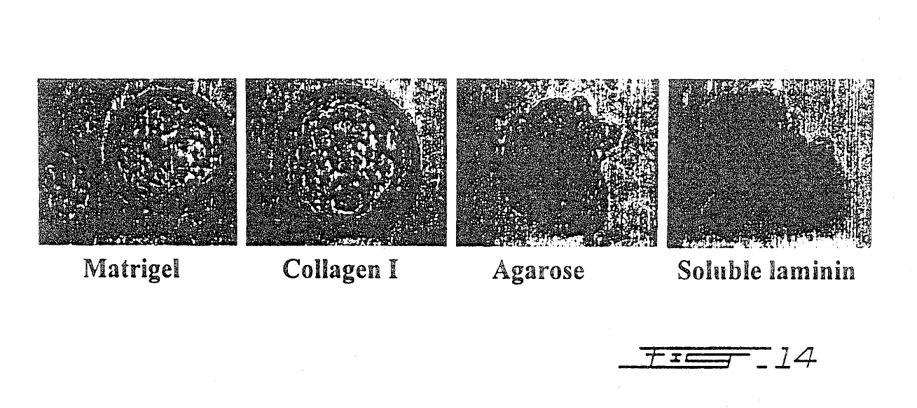

To examine the role of integrin-mediated signaling in the transformation process in a more direct manner, islets were pre-incubated with the RGD motif-containing GRGDSP peptide prior to embedding in collagen. This reduced cystic transformation to 57% of the control DMEM/F12-CT group (p<0.001) at 72 hours (FIG. 14A). The control peptide, GRGESP, had little influence on the transformation process. Pre-treatment islets with either soluble fibronectin or laminin prior to embedding, decreased cystic transformation to 50% of control (p<0.01) at 72 hours (FIG. 14B). Cystic transformation was further reduced to 33% of control, when islets were pre-incubated with both GRGDSP and laminin (p<0.001, FIG. 14C).

Discussion

Differentiated cells usually maintain their cellular specificities in the adult, where stability of cellular phenotype is related to a cell's interaction with its microenvironment. A perturbation or loss of stabilizing factors, however, may induce cells to change their commitment (Okada T S (1986) Develop Growth Diff 28, 213-221). We have reported previously that isolated islets of Langerhans embedded in type 1 collagen gel can be induced to undergo transdifferentiation to duct-like epithelial structures (Yuan S et al. (1996) Differentiation 61, 67-75).

Little is currently known regarding the molecular events involved in transdifferentiation. Hence, the purpose of the present study was to characterize the factors involved in this transformation process in order to better understand the functional relationships that confer morphogenetic stability on cells in the isolated islet. Given the rather poor long-term success rate of cell-based therapies for diabetes mellitus, in particular islet transplantation (Rosenberg L.(1998) Int'l J Pancreatology 24, 145-168), studies such as those described here, could provide new insight into the issues surrounding the problem of graft failure.

There were two principal findings. First, we demonstrated that the process of cystic transformation requires both an elevation of intracellular cAMP and the presence of ECM proteins presented as a solid support. Second, we determined that the formation of a cystic structure from a solid islet sphere is a two-staged process that involves a wave of apoptosis of endocrine cells, followed by cell proliferation of the new duct-like cells.

Signal transduction during transdifferentiation has only recently become the subject of study, therefore detailed information is unavailable. It appears though, that cAMP-mediated information flow plays an important role (Ghee M, Baker H, et al. (1998) Mole Brain Res 55, 101-114; Osaka H, Sabban E L (1997) Mole Brain Res 49, 222-228; Yarwood S J et al. (1998) Mole Cell Endocrinol 138, 41-50). In this study we found that elevation of intracellular cAMP was a necessary, but not a sufficient condition, for induction of islet-to-cyst transformation. However, it was not simply the peak value of the increase in intracellular cAMP that was important, rather it was the duration of the elevation that was associated with the highest frequency of duct epithelial transformation. The increase in cAMP levels, like that produced by medium supplemented with EGF alone or forskolin alone, produced a less than maximal transformation response. The longest duration of cAMP elevation was obtained in medium supplemented with a combination of EGF and CT. This is in keeping with Yao et al. (Yao H, Labudda K, Rim C, et al. (1995) J Biol Chem 270, 20748-20753), who demonstrated the need for sustained versus transient signaling in cAMP-mediated EGF-induced differentiation in PC12 cells. This finding also serves to highlight the similarities between pancreatic β-cells and cells of neuronal origin (Scharfmann R, Czernichow P (1997) Pancreatic growth and regeneration. Pp170-182. Ed Sarvetnick N. Austin: Karger Landes). Therefore, as in other systems (Yao H, Labudda K, Rim C, et al. (1995) J Biol Chem 270, 20748-20753), the cellular responses of islet cells to growth factor action may be dependent not only on the activation of growth factor receptors by specific growth factors, but on synchronous signals that elevate intracellular signals like cAMP.

An increase in intracellular cAMP is of interest too, because a rise in cAMP may form part of the effector system controlling apoptosis in pancreatic β-cells (Loweth A C, Williams G T, et al. (1997) FEBS Lett 400, 285-288). It is therefore noteworthy, that cell loss due to apoptosis is the first step we observed in the process of islet-to-cyst transformation. That apoptosis should occur during islet transformation in this system is interesting, because the islets are embedded in a collagen gel, and such a matrix has been reported to help promote or maintain the differentiated state of different types of cells in culture (Foster CS et al. (1983) Dev Biol 96, 197-216; Yang J et al. (1982) Cell Biol Int 6, 969-975; Rubin K et al. (1981) Cell 24, 463-470). On the other hand, extracellular matrix may also promote the process of transdifferentiation. This point is emphasized by isolated pancreatic acinar cells that transdifferentiate to duct-like structures when entrapped in Matrigel® (Arias A E, Bendayan M (1993) Lab Invest 69, 518-530), and by retinal pigment epithelial cells, which transdifferentiate into neurons when plated onto laminin-containing substrates (Reh T A et al. (1987). Nature 330, 68-71). Most recently, Gittes et al. (Gittes G K et al. (1996) Development 122, 439-447) demonstrated, using 11-day embryonic mouse pancreas, that the default path for growth of embryonic pancreatic epithelium is to form islets. In the presence of basement membrane constituents, however, the pancreatic anlage epithelium appears to be programmed to form ducts. This finding again emphasizes the interrelationship between ducts and islets and highlights the important role of the extracellular matrix. Notwithstanding these observations, the presence of a solid ECM support appears to be a necessary, although not sufficient condition, for the transformation of a solid islet to a cystic epithelial-like structure, the first stage of which, involves apoptotic cell death.

Conversion of a solid to a hollow structure is a morphogenetic process observed frequently during vertebrate embryogenesis (Coucouvanis E, Martin G R (1995) Cell 83, 279-287). In the early mouse embryo, this process of cavitation transforms the solid embryonic ectoderm into a columnar epithelium surrounding a cavity. It has been proposed that cavitation is the result of the interplay of two signals, one from an outer layer of endoderm cells that acts over a short distance to create a cavity by inducing apoptosis of the inner ectodermal cells, and the other a rescue signal mediated by contact with the basement membrane that is required for survival of the columnar cells (Coucouvanis E, Martin G R (1995) Cell 83, 279-287). The combination of these two signals results in death of inner cells not in contact with the ECM and survival of a single layer of outer cells in contact with the basement membrane. A central feature of this model is the direct initiation of apoptosis by an external signal that causes cell death. The second key feature of the model is a signal that appears to be mediated by attachment to ECM and rescues cells from cell death. There is after all, ample precedent for cell dependence on ECM for survival (Meredith J E et al. (1993) Mol Biol Cell 4, 953-961; Boudreau N, Sympson C J, et al. (1995) Science 267, 891-893). In our model of islet-cystic transformation, the external death signal is probably provided by those factors that increase intracellular cAMP. Moreover, the observation that cell loss during the process of transformation occurs preferentially in the center of the islet lends support to the notion that the ECM acts as a rescue signal for those cells in the periphery. The precise role of integrins in this process remains to be more fully delineated. Integrin-ligand binding per se need not contribute to the survival signal. For example, integrins can modulate cell responsiveness to growth factors (Elliot B et al. (1992) J Cell Physiol 152, 292-301).

One area not explored in the present study was the reversibility of the process of transformation. Reversibility of transdifferentiation has been reported in other cell systems (Erenpreisa J, Roach H I. (1996) Mechanisms of Aging & Develop 287, 165-182). Transdifferentiation may involve cell proliferation and the appearance of a multipotential dedifferentiated intermediate cell (Yuan S et al. (1996) Differentiation 61, 67-75) which can express markers characteristic of several alternative phenotypes. It is possible that this is the case in our system (Yuan S et al. (1996) Differentiation 61, 67-75). Thus, it may be possible to expand a population of multipotential cells and then induce guided differentiation to a desired cell phenotype—in this case a mature insulin-producing β-cell. The in-vitro system employed in these studies was unique for two reasons—it did not require fetal tissue, and the starting tissue, adult islets, was well defined.

In summary, this study extends our previous observation that adult islets of Langerhans can be transformed into duct epithelial cystic structures by a two-step process that involves apoptosis followed by cell differentiation and proliferation. The precise biochemical mechanism appears to involve, at least in part, elevation of intracellular cAMP mediated by a combination of cholera toxin and EGF, and a survival signal contributed by a solid ECM support. The differentiation potential of the cells comprising the new epithelial structure remain to be fully elucidated.

### Example III

### Development of a Novel In Vitro Model to Study Acinar-to-Islet Differentiation

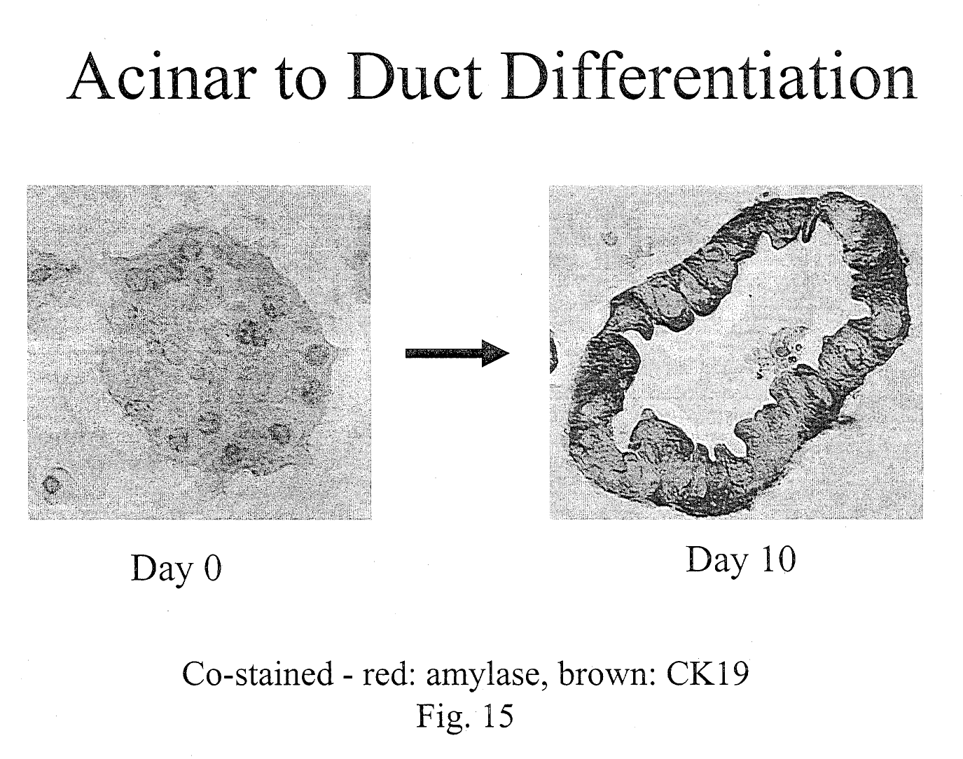

The aim of this experiment was to develop an in vitro model so that the mechanisms and regulatory determinants of acinar to β-cell transformation can be elucidated. Briefly, human donor pancreata (n=3 donors) were cannulated, infused with Liberase HI and digested according to standard protocol. Pancreatic tissue was separated using a continuous ficoll gradient. Acini were isolated from the densest tissue fractions and stained with dithizone to confirm lack of any insulin immunoreactivity. The acinar tissue was then embedded in type-1 rat tail collagen and cultured in DMEM/F12 supplemented with cholera toxin, EGF, and insulin. As determined by inverted microscopy, after 10 days of culture ˜80% of acini transformed into duct-like cystic structures (FIG. 15).

### Example IV

### In situ Duct-Like Structure-to-Islet Differentiation

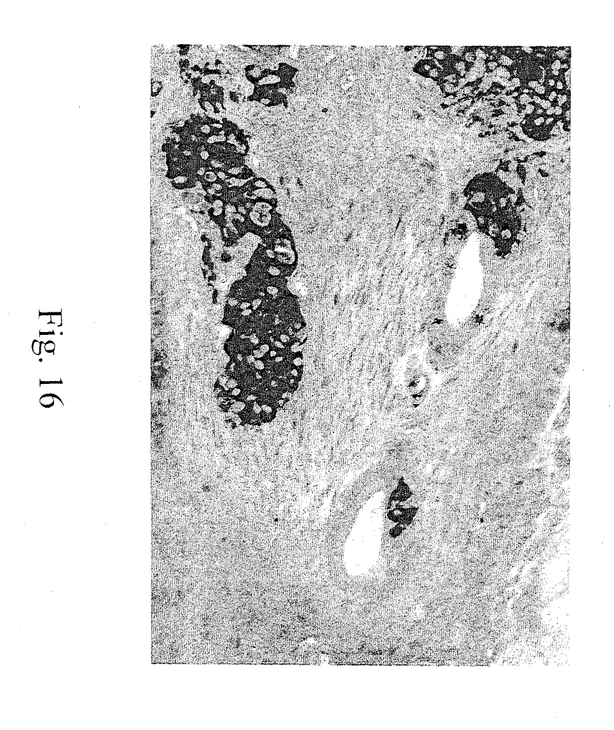

The cystic structures were prepared as described previously. They were combined with differentiated adult islets, pelleted in culture medium by centrifugation and then injected directly into the submucosal space using a fine polyethylene catheter. In the photomicrograph, cells containing insulin are stained red by standard immunocytochemistry. Cells undergoing proliferation are visualized by tritiated thymidine autoradiography, which results in black silver granules overlying the nucleus of the dividing cell (FIG. 16). It can be seen that new insulin-containing cells are differentiating within the wall of duct epithelial structures and that some of these cells are also proliferating.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A medium for preparing duct-like structure cells derived from post-natal islets of Langerhans or acinar cells, which comprises in a physiologically acceptable culture medium an effective amount of:
a) a solid matrix environment for a three-dimensional culture; and
b) a factor for developing, maintaining and expanding said dedifferentiated intermediate cells, said first factor inducing a rise in intracellular cAMP.

2. A medium according to claim 1, wherein said factor is derived from acinar cells.

3. A medium according to claim 1, wherein said culture medium comprises DMEM/12 supplemented with an effective amount of fetal calf serum.

4. A medium according to claim 1, wherein said factor is selected from the group consisting of cholera toxin (CT), forskolin, high glucose concentrations, a promoter of cAMP, and EGF.

5. A medium according to claim 1, wherein said matrix protein comprises one or more of laminin, collagen type I and Matrigel™.

6. A method for preparing duct-like structure cells derived from post-natal islets of Langerhans or acinar cells, which comprises contacting said cells with a medium according to any one of claims 1 to 5.

7. An in vitro method for islet cell expansion, which comprises the steps of:
a) inducing cystic formation in cells cultured in a medium of the present invention, wherein said cells are selected from the group consisting of acinar cells and cells derived from post-natal islets of Langerhans cells to obtain a duct-like structure;
b) expanding cells of said duct-like structure; and
c) inducing islet cell differentiation properties of the expanded cells of said duct-like structure to become insulin-producing cells.

8. An in vitro method for producing cells with at least bipotentiality, which comprises the steps of:
a) inducing cystic formation in cells cultured in a medium of the present invention, wherein said cells are selected from the group consisting of acinar cells and cells derived from post-natal islets of Langerhans cells from a patient to obtain a duct-like structure; whereby when the duct-like structure cells are introduced in situ in the patient, the cells are expanded and islet cell differentiation properties are induced to become in situ insulin-producing cells.

9. A method for the treatment of diabetes mellitus in a patient, which comprises the steps of
a) inducing cystic formation in cells cultured in a medium of the present invention, wherein said cells are selected from the group consisting of acinar cells and cells derived from post-natal islets of Langerhans cells of the patient to obtain a duct-like structure; and
b) introducing the duct-like structure cells in situ in the patient, wherein the cells are expanded in situ and islet cell differentiation properties are induced in situ to become insulin-producing cells.

10. A method for the treatment of diabetes mellitus in a patient, which comprises the steps of
a) inducing cystic formation in cells cultured in a medium of the present invention, wherein said cells are selected from the group consisting of acinar cells and cells derived from post-natal islets of Langerhans cells of the patient to obtain a duct-like structure;
b) expanding in vitro the duct-like structure cells;
c) inducing in vitro islet cell differentiation properties of the expanded cells of duct-like structure to become insulin-producing cells; and
d) introducing the cells of step c) in situ in the patient, wherein the cells produce insulin in situ.
